(19) United States
(12) Patent Application Publication
(10) Pub. No.: US 2012/0041185 A1
(43) Pub. Date: Feb. 16, 2012

(54) dTDP-BETA-D-FUCOFURANOSE, ITS PREPARATION METHOD AND USE

(75) Inventors: Lei Wang, Tianjin (CN); Quan Wang, Tianjin (CN); Lu Feng, Tianjin (CN)

(73) Assignee: TIANJIN BIOCHIP TECHNOLOGY CORPORATION, Tianjin (CN)

(21) Appl. No.: 12/672,349

(22) PCT Filed: Sep. 14, 2007
(86) PCT No.: PCT/CN07/02721

§ 371 Date: Sep. 6, 2011

(30) Foreign Application Priority Data
Sep. 3, 2007 (CN) 200710145609.8

(51) Int. Cl. C07H 19/10; C12P 19/02

(52) U.S. Cl. 536/26.23; 435/105

(57) ABSTRACT

Provided is a dTDP-β-D-fucofuranose, which is also referred to as dTDP-β-6-deoxy-D-galactofuranose. The dTDP-β-D-fucofuranose is synthesized by using reductase Fcf1 and mutase Fcf2 in the gram-negative bacteria. Also provided are the preparation method of the dTDP-β-D-fucofuranose and use of the dTDP-β-D-fucofuranose for manufacturing a medicament for the treatment of tumors.

### FIELD OF TECHNOLOGY

The subject matter of the disclosed invention is related to the enzymatic synthesis of gram negative bacterial surface monosaccharide, especially the reagents, reagent kits and methods of dTDP-D-fucofuranose biosynthesis in Escherichia coli O52 as well as the saccharide application.

### BACKGROUND OF THE INVENTION

Carbohydrates, one of the most abundant biological material in nature, exercise numerous biological functions. They store energy, maintains cell structure, constitute the extracellular components, exercise the cell-to-cell signal recognition and transduction etc. Carbohydrates constitute structure basis of important informational molecules in the daily life activities (growth, hemotype, maintenance of the nervous system and immune system) or medical application (organ transplant, inflammation, autoimmune disease, aging, cancer cell proliferation and metastasis, pathogen infection) or the plants and pathogens interaction. Carbohydrates, always exposed on the cell surface or linked to secretary proteins, play a great role in cell-to-cell recognition, modulation, communication and signal transduction.

The cell wall of gram-negative bacteria can be conceptually divided into three layers: lipoproteins, outer membrane and lipopolysaccharide (LPS) from inside to outside. LPS contains three parts: lipid A, core polysaccharides and O-specific polysaccharide chain (O-antigen). O-antigen linked to the core polysaccharide lies in the outer leaflet of the outer membrane and constitutes the main antigen of cell surface. The O-antigen repeats usually contain two to eight monosaccharide units and all of them take on the precursor form—NAD-monosaccharide to composite O-antigen. These monosaccharides usually can be divided into two types, one type is these common monosaccharide and their derivatives in cell metabolism; the other type is the rare monosaccharide specially in O-antigen including many monosaccharides, their derivatives, analogues, etc. And most of them are produced by multiply enzyme reactions on the first type monosaccharide.

Rare monosaccharides exist in all sorts of biological macromolecules such as glycoproteins, glycolipids, bacterial secondary metabolites including antibiotics and they act an irreplaceable role to their bioactivity. Particularly some of their preparation are relevant to the synthesis and activity of antibiotics. Rare monosaccharides in animals and human being can render intense immune response. We can ferment those rare monosaccharides, affect the activity of the biological molecules by modifying saccharide's structure or create new carbohydrate using recombination technology. This technology can be used to produce new antibiotics. In recently years, many new medicines produced by genetic engineering come into being. For example, two new types of antibiotics made by modifying saccharide or synthesizing new saccaride have been successfully developed and applied in treatment.

Since the rapid development of genomics, lately, more than 100 polysaccharides gene clusters have been deciphered. Moreover, gene function and synthesis pathway of common monosaccharides and part of rare saccharides are well-established. (See http://www.microbio.usyd.edu.au/BPGD/default.htm).

Carbohydrates have high complexity and diversity. Only part of the monosaccharides can be produced by chemical synthesis due to the high cost. The synthesis of rare saccharides with chemical method encounters many difficulties, while the rare monosaccharides associated with life are extremely difficult to purify. Therefore mass production of rare monosaccharides has been the most important target in the saccharide research for years. Biosynthesis of saccharides is the most promising way. Utilizing combinations of function well-known synthetase to produce inexistent or important rare monosaccharide means a lot to medical treatment and biological pharmacy.

dTDP-D-fucofuranose is the precursor of monosaccharide D-fucofuranose which has ever been proved to the sugar moiety of the anticancer drug—gilvocarcin V. D-fucofuranose possesses important biological activity.

Up to now, dTDP-D-fucofuranose biosynthesis pathway in E. coli O52 and its biological characterization haven't been reported.

### SUMMARY OF THE INVENTION

The invention provides dTDP-D-fucofuranose with conformation (I).

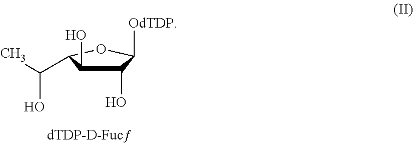

The above-described dTDP-D-fucofuranose is synthesized by Fcf1 reductase and Fcf2 mutase in Gram-negative bacterium.

Another purpose of this invention is to illustrate the biosynthesis pathway of dTDP-D-fucofuranose. The main steps are listed as follows:
(1) RmIA transferase catalyses the conversion of D-Glc-1-P to dTDP-D-glucose (dTDP-D-Glc).
(2) RmIB dehydrase catalyses the conversion of dTDP-D-Glc to TDP-6-deoxy-D-xylo-hex-4-ulopyranose (dTDP-D-GIcO).
(3) Fcf1 catalyses the conversion of dTDP-6-deoxy-D-xylo-hex-4-ulopyranose to dTDP-D-fucopyranose (dTDP-D-Fucp).
(4) Fcf2 converts dTDP-D-Fucp into dTDP-D-Fucofuranose.

After a great deal of research and creative work, we work out this program-firstly to produce dTDP-D-Fucp, in this foundation, synthesize dTDP-D-Fucofuranose.

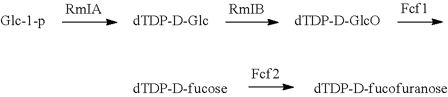

As referred above,

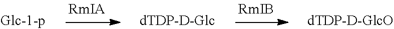

is available technology,
otherwise,

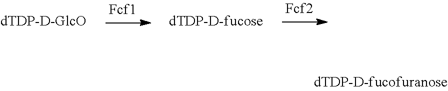

is our original work containing our massive painstaking care.

The above mentioned dTDP-D-fucose have conformation (II).

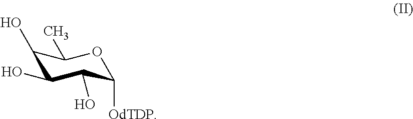

The characterization of reductase Fcf1 coding genes are among the following nucleotide sequence a), b) or c).
a) nucleotide sequence SEQ ID NO:1.
b) Because of the degeneracy of genetic code, the gene coding sequences can be different from SEQ ID NO:1, but they must encode the same amino acids.
c) The coding genes can hybrid with sequence a) or b) under strict hybridization condition and produce an active Fcf1.
   Reductase Fcf1 have the same amino acids as the following g), h) or i).
g) amino acids encoded by a), b) or c).
h) amino acids encoded by SEQ ID NO:3.
i) The sequences derive from deletion, replacement or insertion one or more amino acids of the amino acid sequences encoded by SEQ ID NO:3 which can also produce active reductases.
   The coding sequence of mutase Fcf2 has the following characterization listed in d), e) or f).
d) nucleotide sequence SEQ ID NO:2.
e) Because of the degeneracy of genetic code, the gene coding sequences can be different from SEQ ID NO:2, but they must encode the same amino acids.
f) The coding genes can hybrid with sequence d) or e) under strict hybridization condition and produce an active Fcf2.
   the amino acid sequences of Fcf2 must meet the requirements presenting in j), k) or l).
j) amino acid encoded by the above d), e) or f).
k) amino acids encoded by SEQ ID NO:4.
l) The above-mentioned amino acid sequence encoded by SEQ ID NO:4 can be deleted, replaced or inserted one or more amino acids and the proteins encoded still possess the Fcf2 activity.

A new recombinant plasmid expressing reductase Fcf1 is constructed in step (3) and cloned into pET28a (+) expression vector.

As previously mentioned a recombinant strain is constructed to produce reductase Fcf1 in step (3).

As referred before the plasmid pET28a (+) containing Fcf2 encoding sequence is constructed in step (4).

Accordingly, we also construct an expression strain containing Fcf2 to express target proteins in step (4).

It should be noted that the stated term “stringent hybridization condition” refers to specific condition under which only specific hybridization can be formed while non-specific hybridization can not be formed. For example, the stringent hybridization could be defined as different DNA sequences with similarity of not less than 70 percent can hybridize while DNA sequences can not produce hybridization the similarity of which below the above mentioned value. And that DNA sequences not less than 90 percent similarity could hybridize would be the optimal choice. Relative to the ordinary washing condition in Southern blotting, we utilize the following condition: place the hybridization filter in the pre-hybridization buffer (0.25 mol/L sodium phosphate buffer pH7.0, 7% SDS), hybridization for 30 min under 50° C. Then discard the pre-hybridization solution, add hybridization buffer (0.25 mol/L sodium phosphate buffer pH7.0, 7% SDS, Isotope-labeled nucleotide fragments). Hybridization is carried for 12 hours at 50° C. Discard the hybridization solution, add membrane wash buffer I (2×SSC and 0.1% SDS) and wash twice at 50° C., each time for 30 min. Add the membrane wash buffer II (0.5×SSC and 0.1% SDS) and incubate at 50° C. for 30 min.

As is known to all in this field, these amino acid sequences derived from some modifications of the translation of the Fcf1 and Fcf2 encoding genes defined by SEQ ID NO:1, SEQ ID NO:2, such as insertion one or more amino acids, insertion, deletion, can also produce active proteins and their coding sequences should have been considered as Fcf1 and Fcf2 encoding genes.

Furthermore, those active proteins with one or more amino acids insertion or deletion of the product translated from SEQ ID NO:1, SEQ ID NO:2 (Fcf1 and Fcf2 encoding genes) are satisfied our requirements and deemed as our purpose as well. Therefore, our invention includes the following proteins with Fcf1, Fcf2 activity whose amino acid similarity with SEQ ID NO:3 and SEQ ID NO:4 is at least 70 percent. What's more, functional proteins with similarity value above 90 percent is our preference. The term “more” above mentioned can be less than 100, but less than 10 is the optimal choice.

dTDP-D-fucofuranose application is also involved in this invention.

For instance, dTDP-D-fucofuranose can be used in the field of medicine as anticancer drug for its excellent biocompatible form in vivo. “biocompatible form in vivo” refers to a material form that can reduce any toxicity during treatment. Living organism including animals and human being can take the medicine. The effective doses of the drug ingredients means drug in certain concentration can produce a desired result over sufficient time. For example, a drug effective dose can be affected by many factors like patient's condition, age, gender, weight and the effect of injected antibodies. Otherwise take a daily dose throughout the day or decrease the does of medicine according to the emergency in treatment.

Appropriate methods of drug use include injection (subcutaneous injection, intravenous injection), oral administration, inhalation, or rectal absorption. According to different methods of drug administration, effective constituent can be wrapped to avoid the affection of enzymes, acids and other substances which could inactivate them.

“Medicine” here above can be produced by ever known drug preparation methods, that is, mix the active constituent in effective does with treatment media together. Many medias have ever been described, such as Remington's Pharmaceutical Sciences (Remington's Pharmaceutical Sciences, Mack Publishing Company, Easton, Pa., USA 1985). Besides, the soluble ingredient within the drug can mix with one kind or multiple treatment reagents or their dilution dissolved in physiological buffer under the appropriate pH condition and ionic osmotic pressure.

The sugar can also be used as merchandise or reaction reagent.

In order to make the above and other purpose, feature and advantage of this invention more clear and understandable, here we give several implementation of special cases together with attached figures. More instructions in detail as follows.

### BRIEF DESCRIPTION OF THE DRAWINGS

For a more complete understanding of this invention, reference should now be made to the accompanying drawings and described below by way of examples of the invention wherein:

FIG. 1: Capillary electrophoresis chromatogram of Fcf1 and Fcf2 reactions

FIG. 2: MS spectra of dTDP-D-fucose product

FIG. 3: MS/MS spectra of dTDP-D-fucose product

FIG. 4: MS/MS/MS spectra of dTDP-D-fucose product

FIG. 5: MS spectra of dTDP-D-fucofuranose product

FIG. 6: MS/MS spectra of dTDP-D-fucofuranose product

FIG. 7: MS/MS/MS spectra of dTDP-D-fucofuranose product

FIG. 8: SDS-PAGE analysis of purified protein Fcf1

FIG. 9: SDS-PAGE analysis of purified protein Fcf2

### DETAILED DESCRIPTION OF THE DISCLOSED INVENTION

Details of the invention will be explained by combining operation examples with figures attached, and what should merit is that the following operation examples are just for illustrations but not for restriction of the invention.

### Operation Example 1

### Clones of Reductase Fcf1 and Mutase Fcf2

1. Genomic DNA Extraction of E. coli O52

Centrifuge 3 ml overnight bacterial culture, discard the supernatant. The pellet is re-suspended in 250 μl Tris buffer (50 mM, pH 8.0). Centrifuge and remove the supernatant. Resuspend the pellet in 250 μl 50 mM Tris buffer (pH 8.0) plus 10 μl 0.4M EDTA (pH 8.0). Mix and incubate at 37° C. for 20 minutes; 15 μl lysozyme 20 mg/ml is added and blend it well, then incubate at 37° C. for 15 min. Add 2 μl of 50 mg/ml of protease K solution, blend gently, and then put in 15 μl 10% SDS, incubate at 50° C. till the suspension becomes clear; Add 7 μl of 25 mg/ml of RNAase buffer, incubate for 15 minutes at 65° C.; Extract with equal volume phenol:chloroform:isoamyl alcohol (1:23:1) twice and chloroform:isoamyl alcohol (23:1) once. Transfer upper clear supernatant to new tube and pour 2 volumes of cold ethanol and mix gently to precipitate DNA. After centrifugation wash well with 70% ethanol. The pellet is dried and dissolved in 20 μl TE (pH 8.0, 10 mM Tris, 1 mM EDTA).

2. Reductase Fcf1 Clone Construction and Screening

Fcf1 is amplified from E. coli O52 genomic DNA by PCR using the following primer pairs: oligonucleotide sequence of SEQ ID NO: 5/SEQ ID NO: 6. A total of 30 cycles are performed using the following conditions:

the PCR cycle parameter:

A 3-min denaturation is carried out followed by 30 cycles of 15 s at 94° C., 30 s at 50° C. and 1 min at 72° C. A final elongation step of 5 min at 72° C. is performed. Then 4° C. for 2 hours.

PCR product is digested with EcoRI and XhoI. 0.8% agarose gel electrophoresis is processed. Cut gel about 0.9 kb and purify the digested PCR product. Ligate the PCR product with plasmid pET-28a(+) digested with same restriction enzymes; pET-28a (+) is purified by gel extraction kit after digestion as well. The ligation product is transformed into E. coli DH5α competent cell by electroporation. Then smear on the solid LB broth with Kan (Kanamycin) of 50 μg/ml. Cultivate at temperature 37° C. for 12 hours. Select colonies, extract their plasmids and verify with restriction enzymes. The pET-28a(+) with SEQ ID No:1 inserted is recombinant plasmid plw1203, and the DH5a including the plasmid is recombinant strain H1441. plw1203 is sequenced with Sanger chain-terminator method. The sequence result shows that the DNA fragment from initiation codon ATG to stop condon TAA is 951 bp the same as nucleotide sequence defined by SEQ ID No:1. The entire ORF encodes a protein composed of 316 amino acids, which belongs to the NAD-depended isomerase family or anhydrase family. And the similarity is not high on even by blast. Amongst the UDP-glucose-4-epimerase of Prochlorococcus marinus shows a highest similarity with Fcf1. The similarity value is 33% and identity is 55%.

3. Mutase Fcf2 Cloning and Screening

Fcf2 is amplified from E. coli O52 genomic DNA by PCR using the primer pairs: oligonucleotide sequence of SEQ ID NO: 7/SEQ ID NO: 8. A total of 30 cycles were performed using the following conditions:

The PCR cycle parameter:

A 3-min denaturation is carried out followed by 30 cycles of 15 s at 94° C., 30 s at 50° C. and 1 min at 72° C. A final elongation step of 5 min at 72° C. is performed. Then 4° C. for 2 hours.

Both the PCR product and the pET-28a vector are digested with EcoRI and XhoI, purified using gel extraction kit. The PCR product is at position of 1.1 kb. Ligate the PCR product with plasmid pET-28a(+) digested using T4 ligase; The ligation product is transformed into E. coli DH5α competent cell by electroporation. Then smear on the solid LB broth with Kan (Kanamycin) of 50 μg/ml. Cultivate at temperature 37° C. for 12 hours. Select mono colonies, extract their plasmids and verify with restriction enzymes. The pET-28a(+) with SEQ ID No:2 inserted is recombinant plasmid plw1204, and E. coli DH5α including the plasmid is recombinant strain H1442. plw1204 is sequenced with Sanger chain-terminator method. The sequence result shows that the DNA fragment from initiation codon ATG to stop condon TAA is 1134 bp overall the same as nucleotide sequence defined by SEQ ID No:2. The entire ORF encodes a protein composed of 377 amino acids, which belongs to the UDP-fucofuranose mutase family. Fcf2 shares the most similarity with UDP-fucofuranose mutase in Klebsiella pneumoniae and the value is 60 percent.

### Operation Example 2

### Purification of Protein Fcf1 and Fcf2

1. Purification of Hisₛ-Fcf1

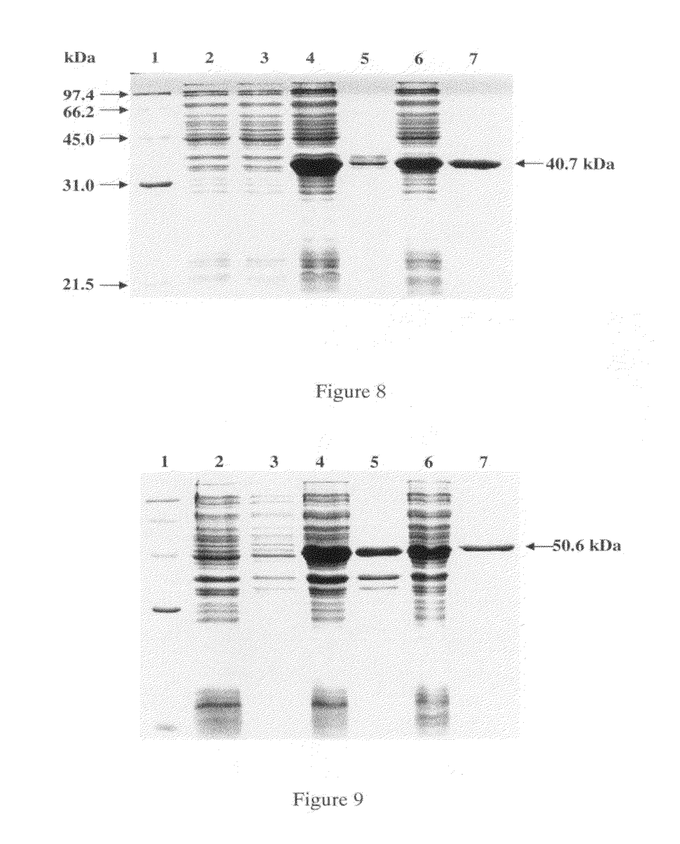

Extract the plasmid plw1203 from above mentioned E. coli DH5α H1441 and transfer it into expression strain E. coli BL21 and screen the positive transformant; inoculate the transformant monoclone into 20 ml LB broth containing Kan of 50 μg/ml, incubate at 200 rpm and 37° C. for 12 h. For the expression of His6-Fcf2, 250 ml of LB broth (two culture bottles) containing kanamycin (50 μg/ml) is inoculated with the ratio 1% (V/V) of an overnight culture, and grown at 37° C. 220 rpm. When OD_{600 nm} reach 0.6, IPTG is added to a final concentration of 0.1 mM, and expression was allowed to proceed for 4 h at 25° C., 180 rpm. Cells are harvested by centrifugation. The pellet is resuspended in volumes of Binding buffer (50 mM Tris-HCl pH 8.0, 300 mM NaCl, 10 mM imidazole). The cells are disrupted by ultrasonication on ice, and cell debris and membrane fractions are removed by ultracentrifugation. The supernatant is crude extraction of dTDP-D-GIGO reductase Fcf1. The His6-tagged fusion proteins in the supernatant are purified by nickel ion affinity chromatography with a Chelating Sepharose Fast Flow column (GE Healthcare) according to the manufacturer's instruction. The purified proteins get one band on SDS-PAGE analysis. Assay the recombined dTDP-GlcO reductase with protein marker, and deduce its molecular weight of 40738 dalton, similar with the theoretical calculation (39416 dalton). Just as is indicated by FIG. 8: 1. protein Marker; 2. pET28a vector; 3. total protein in pET28a with DNA insertion before induction; 4. total protein of pET28a with DNA insertion after induction; 5. protein in pellet following ultracentrifugation after induction; 6. soluble cell protein after induction; 7. recombinant protein after elution.

2. Purification of Mutase Fcf2

Extract the plasmid plw1204 from above mentioned E. coli DH5α H1442 and transfer it into expression strain E. coli BL21 and screen the positive transformant; inoculate the transformant monoclone into 20 ml LB broth containing Kan of 50 μg/ml, incubate at 200 rpm and 37° C. for 12 h. For the expression of His6-Fcf2, 250 ml of LB broth (two culture bottles) containing kanamycin (50 μg/ml) is inoculated with the ratio 1% (V/V) of an overnight culture, and grown at 37° C. 220 rpm. When OD_{600 nm} reach 0.6, IPTG is added to a final concentration of 0.1 mM, and expression was allowed to proceed for 4 h at 22° C., 220 rpm. Cells are harvested by centrifugation. The pellet is resuspended in volumes of Binding buffer (50 mM Tris-HCl pH 8.0, 300 mM NaCl, 10 mM imidazole). The cells are disrupted by ultrasonication on ice, and cell debris and membrane fractions are removed by ultracentrifugation. The supernatant is crude extraction of dTDP-D-fucofuranose mutase Fcf2. The His6-tagged fusion proteins in the supernatant are purified by nickel ion affinity chromatography with a Chelating Sepharose Fast Flow column (GE Healthcare) according to the manufacturer's instruction. The purified proteins get one band on SDS-PAGE analysis. Assay the recombined dTDP-GIcO aminotransferase with protein marker, and deduce its molecular weight of 50580 dalton, similar with the theoretical calculation (48015 dalton). Just as is indicated by FIG. 9: 1. protein Marker; 2. pET28a vector; 3. total protein in pET28a with DNA insertion before induction; 4. total protein of pET28a with DNA insertion after induction; 5. protein in pellet following ultracentrifugation after induction; 6. soluble cell protein after induction; 7. recombinant protein after elution.

### Operation Example 3

### Detection of dTDP-D-fucose and dTDP-D-fucofuranose in E. coli O52

1. Detection of dTDP-D-fucose

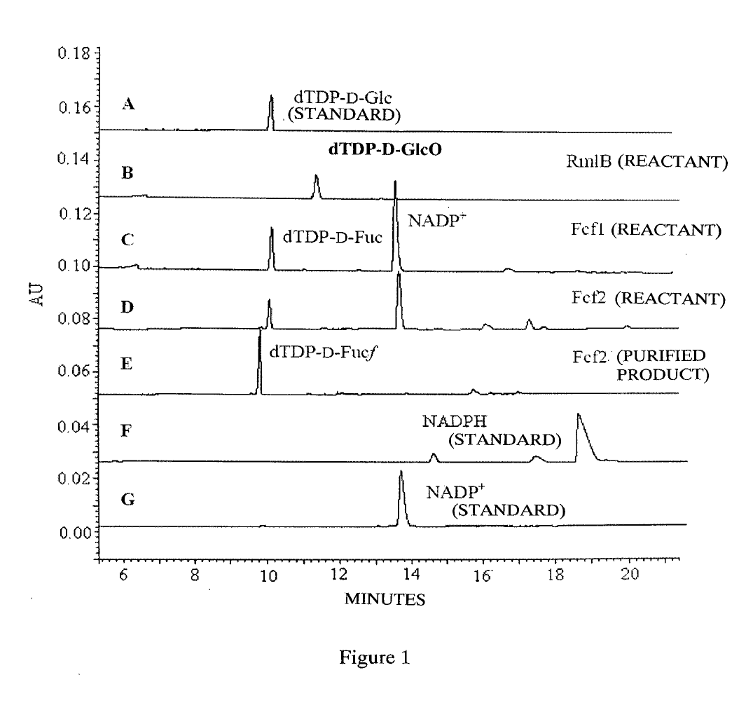

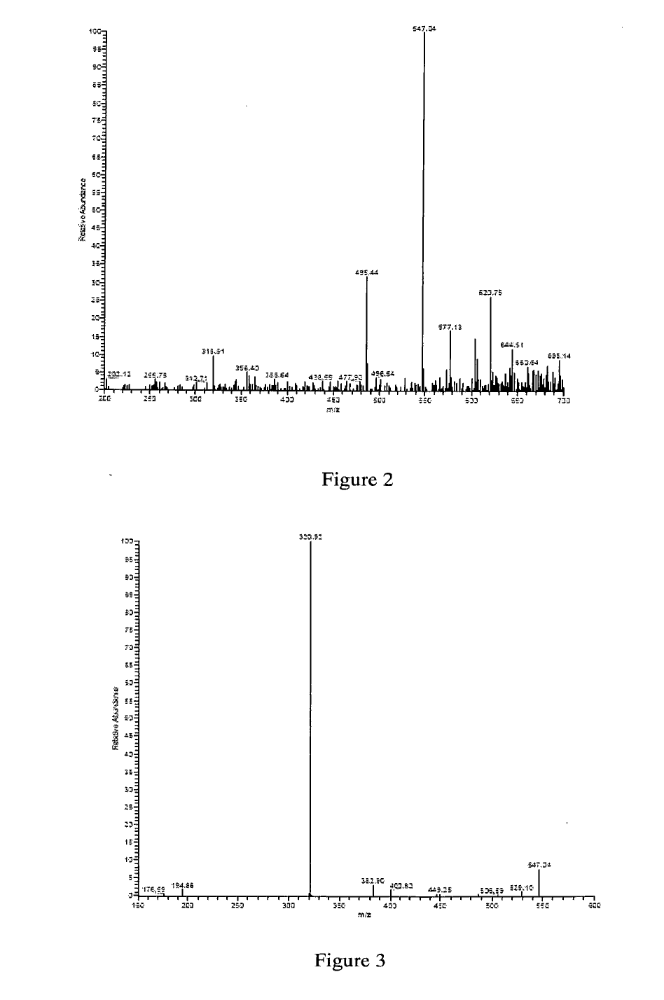

20 μl reactions in 0.5 ml centrifugal tube contain 2 mM dTDP-D-Glco, 3 mM NADPH, 50 mM Tris-HCl buffer (pH7.4) and 0.25 μM recombinant dTDP-D-GIcO reductase protein purified in example 2. The reactions are proceeded at 37° C. for 2 hours. Then add chloroform of equal volume to extract protein. The aqueous phase is detected by Beckman Coulter P/ACE MDQ capillary electrophoresis and the result is indicated by C in FIG. 1, which shows that substrate disappears and new product appears (B in FIG. 1 is dTDP-GIcO, product of anhydrase). Repeat the reaction till 500 μl accumulation, detect with Finnigan LCQ Advantage MAX mass spectrometer and identify it initially as dTDP-D-fucose, as indicated in FIG. 2.

2. Detection of dTDP-D-fucofuranose

25 μl reactions in 0.5 ml centrifugal tube contain 2 mM dTDP-D-fucose, 50 mM Tris-HCl buffer (pH7.4) and 3.9 μM recombinant dTDP-D-Glcfucofuranose mutase protein purified in example 2. The reactions are proceeded at 37° C. for 3 hours. Then add chloroform of equal volume to extract protein. The aqueous phase is detected by Beckman Coulter P/ACE MDQ capillary electrophoresis and the result is indicated by D in FIG. 1, which shows new product appears together with original reagents. Repeat the reaction till 500 μl accumulation, detect with Finnigan LCQ Advantage MAX mass spectrometer and identify it initially as dTDP-D-fucofuranose, as indicated in FIG. 5.

### Operation Example 4

### Separation, Purification and Electro-Spray Ionization Mass Spectrometry Detection of dTDP-D-Fucose & dTDP-D-Fucofuranose

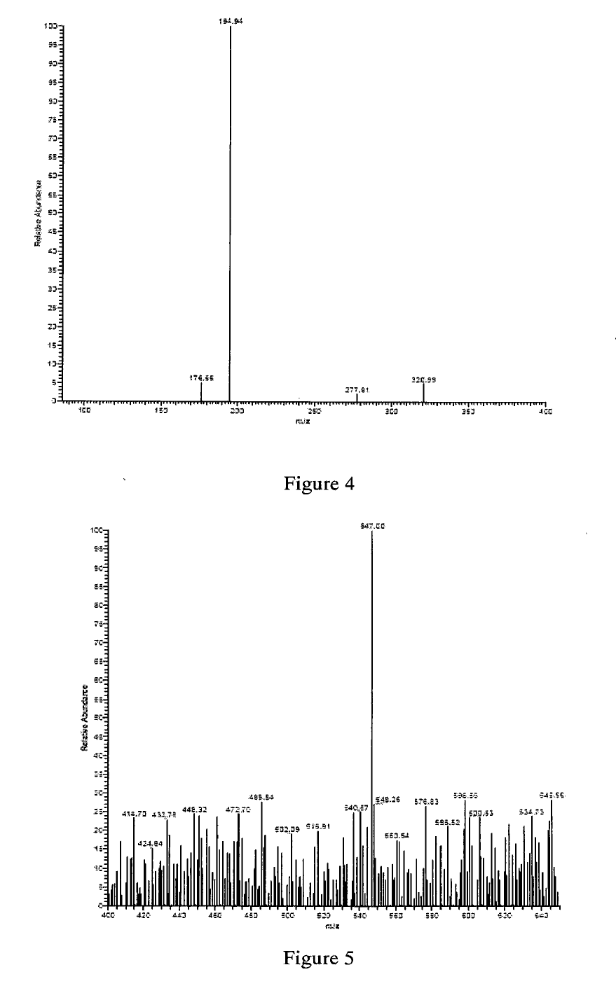

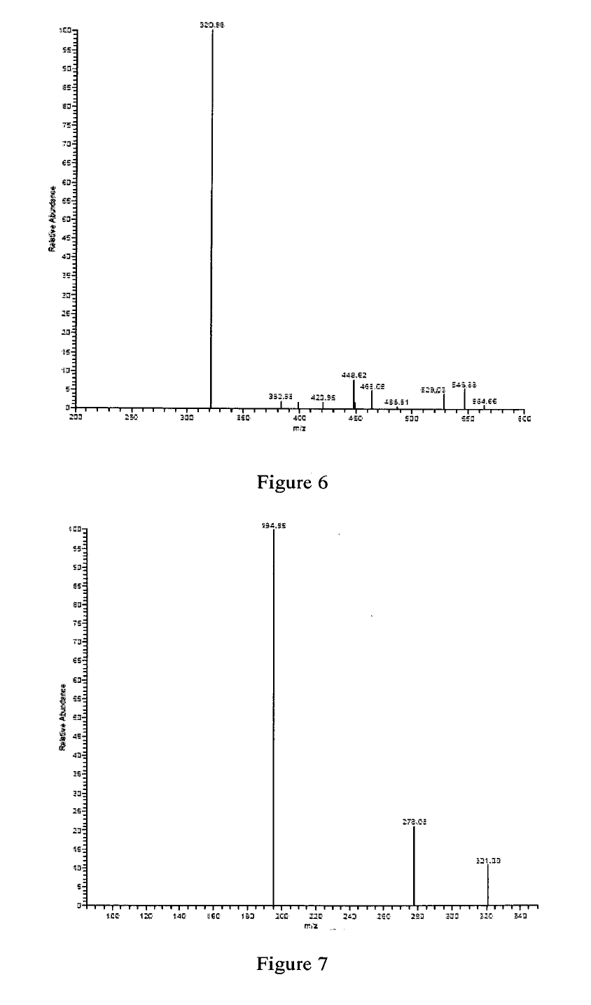

The Fcf1 and Fcf2 reaction products are purified by reverse-phase HPLC using a BioCAD 700E Perfusion Chromatography Workstation (Applied Biosystems, CA) with aVenusil MP-C18 column (5 mm particle, 4.6 mm×250 mm). The mobile phase used is composed of 3.3% of acetonitrile and 96.7% of 50 mM triethylamineacetatic acid (pH 6.8). The flow rate is 0.6 ml/min. Fractions containing expected products are collected, lyophilized and re-dissolved in 50% of methanol before injecting into a Finnigan LCQ Advantage MAX ion trap mass spectrometer (Thermo Electron, CA) at negative mode (4.5 kV, 250° C.) for ESI-MS analysis. For MS2 and MS3 analyses, nitrogen is used as collision gas and helium as auxiliary gas and collision energies used are typically 20-30 eV. MS/MS and MS/MS/MS results of dTDP-D-fucose are respectively indicated by FIG. 3 and FIG. 4, while MS/MS and MS/MS/MS results of dTDP-D-fucofuranose are respectively indicated by FIG. 6 and FIG. 7.

### Operation Example 5

### Confirmation the Pathway for Synthesis of dTDP-D-Fucofuranose in E. coli O52

RmIA and RmIB of the dTDP-D-fucofuranose synthetic pathway in E. coli O52 have been confirmed in other bacterial strains, with over 65% similarity. Through CE detection, the inventor thinks that the function of the above mentioned enzymes in E. coli O52 is to convert Glc-1-P into dTDP-GIcO. Fcf1 is confirmed to be a dTDP-6-deoxy-D-xylo-hex-4-ulopyranose reductase for the conversion of dTDP-6-deoxy-D-xylo-hex-4-ulopyranose to dTDP-D-fucopyranose (dTDP-D-Fucp), and Fcf2 a dTDP-D-Fucp mutase for the conversion of dTDP-D-Fucp to dTDP-D-Fucf in example 3. Hereof, synthetic pathway of dTDP-D-fucofuranose in E. coli O52 can be stated like this:

Glc-1-p-dTDP-D-Glc-dTDP-D-GlcO-dTDP-D-fucose-dTDP-D-fucofuranose.

### Operation Example 6

### Enzyme Activity Assays

1. Enzyme Activity Assay of Fcf1

20 μl reactions in 0.5 ml centrifugal tube contain 2 mM dTDP-D-Glco, 3 mM NADPH, 50 mM Tris-HCl buffer (pH7.4), 5 mM divalent ion chloride and 0.25 μM recombinant dTDP-D-GIcO reductase protein purified in example 2. The reactions are proceeded at certain temperatures for 2 hours. Then add chloroform of equal volume to extract protein. The aqueous phase is detected by Beckman Coulter P/ACE MDQ capillary electrophoresis.

(1) Determination of Temperature Optima

To determine the temperature optima for Fcf1, standard reactions are carried out at 4° C., 15° C., 25° C., 37° C., 50° C., 65° C. and 80° C. respectively and the result shows that the optimum temperature for the enzyme activity is 15-37° C.

| | temperature (° C.) | | | | | | |
|---|---|---|---|---|---|---|---|
| | 4 | 15 | 25 | 37 | 50 | 65 | 80 |
| transformation rate | 47.0 | 100 | 100 | 100 | 35.5 | 31 | 22 |
| (%) | | | | | | | |

(2) Determination of Divalent Cation Requirements

To test effects of different cations on the enzyme activities, standard reactions are carried out in the presence of 5 mM MgCl₂, MnCl₂, FeCl₂, CuCl₂, CaCl₂, CoCl₂ respectively and the result shows that the inhibition ability is Ca²⁺, Fe²⁺, Co²⁺, Cu²⁺ low to high, among which Mg²⁺ and Mn² have no influence to conversion ratio, and Cu²⁺ has the highest inhibition to enzyme activity.

| | | ions | | | | | |
|---|---|---|---|---|---|---|---|
| | none | Ca²⁺ | Co²⁺ | Cu²⁺ | Fe²⁺ | Mn²⁺ | Mg²⁺ |
| Transformation | 100 | 98.1 | 89.2 | 11.8 | 90.5 | 100 | 100 |
| rate (%) | | | | | | | |

(3) Measurement of Kinetic Parameters

In the condition of the foregoing reaction (37° C., 3 mM MADPH, 50 mM Tris-HCl buffer (pH 7.4), 0.25 μM enzyme, 30 seconds), the concentration of dTDP-GIcO is 0.1-1 mM. Measure the concentration of dTDP-Qui4N. The Kₘ of Fcf1 is 0.54 mM and k_{cat} is 956 min⁻¹ calculated based on the Michaelis-Menten equation.

### 2. Enzyme Activity Assay of Fcf2

25 μl reactions in 0.5 ml centrifugal tube contain 2 mM dTDP-D-fucose, 50 mM Tris-HCl buffer (pH 7.4) 5 mM divalent ion chloride and 3.9 μM recombinant dTDP-D-Glcfucofuranose mutase protein purified in example 2. The reactions are proceeded at certain temperature for 3 hours. Then add chloroform of equal volume to extract protein. The aqueous phase is detected by Beckman Coulter P/ACE MDQ capillary electrophoresis.

(1) Determination of Temperature Optima

To determine the temperature optima for Fcf2, standard reactions are carried out at 4° C., 15° C., 25° C., 37° C., 50° C., 65° C. and 80° C. respectively and the result shows that the optimum temperature for the enzyme activity is 37° C.

| | Temperature (° C.) | | | | | | |
|---|---|---|---|---|---|---|---|
| | 4 | 15 | 25 | 37 | 50 | 65 | 80 |
| Transformation | 1.6 | 9.5 | 9.6 | 9.6 | 4.7 | 0 | 0 |
| rate (%) | | | | | | | |

(2) Determination of Divalent Cation Requirements

To test effects of different cations on the enzyme activities, standard reactions are carried out in the presence of 5 mM Mg²⁺, Mn²⁺, Fe²⁺, Cu²⁺, Ca²⁺, Co²⁺ respectively and the result shows that only Cu²⁺ has obvious inhibition to enzyme activity, but other metal ions have no obvious inhibition.

| | | ions | | | | | |
|---|---|---|---|---|---|---|---|
| | none | Ca²⁺ | Co²⁺ | Cu²⁺ | Fe²⁺ | Mn²⁺ | Mg²⁺ |
| Transformation | 9.5 | 9.6 | 8.8 | 0 | 9.1 | 5.4 | 9.7 |
| rate (%) | | | | | | | |

Although a few good operation examples of the invention are uncovered ibid, they shall not restrict the invention. Any technician in the field, without violating the invention spirit and range, may do certain changes and improvements. Therefore protection scope of the invention should be subject to the definition of the inventor.

What is claimed is:

1. dTDP-D-Fucofuranose, the specificity lying in the structure conformation (I):

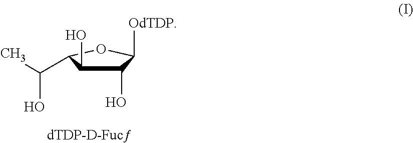

2. An enzymatic synthesis method of dTDP-D-Fucofuranose of claim 1 comprising the steps of:
(1) RmIA catalyses the conversion of D-Glc-1-P to dTDP-D-glucose (dTDP-D-Glc);
(2) RmIB catalyses the conversion of dTDP-D-Glc to TDP-6-deoxy-D-xylo-hex-4-ulopyranose;
(3) Fcf1 catalyses the conversion of dTDP-6-deoxy-D-xylo-hex-4-ulopyranose to dTDP-D-fucopyranose (dTDP-D-Fucp).
(4) Fcf2 functions as dTDP-D-Fucp mutase for the conversion of dTDP-D-Fucp to dTDP-D-Fucofuranose.

3. The method as defined in claim 2 wherein dTDP-D-fucopyranose has the conformation (II):

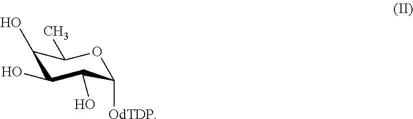

4. The method as defined in claim 2 wherein the characterization of reductase Fcf1 coding genes are among the following nucleotide sequence a), b) or c):
a) nucleotide sequence SEQ ID NO:1;
b) because of the degeneracy of genetic code, the gene coding sequences can be different from SEQ ID NO:1, but they must encode the same amino acids; and
c) the coding genes can hybrid with sequence a) or b) under strict hybridization conditions and produce an active Fcf1.

5. The method as defined in claim 2 wherein reductase Fcf1 have the same amino acids as the following g), h) or i):
g) amino acids encoded by a), b) or c);
h) amino acids encoded by SEQ ID NO:3;
i) the sequences derive from deletion, replacement or insertion of one or more amino acids of the amino acid sequences encoded by SEQ ID NO: 3 which can also produce active reductases.

6. The method as defined in claim 2 wherein the coding sequence of mutase Fcf2 has the following characterization listed in d), e), or f):
d) nucleotide sequence SEQ ID NO:2;
e) because of the degeneracy of genetic code, the gene coding sequences can be different from SEQ ID NO:2, but they must encode the same amino acids;
f) the coding genes can hybrid with the sequence d) or e) under strict hybridization conditions and produce an active Fcf2.

7. The method as defined in claim 2 wherein the amino acid sequences of Fcf2 must meet the requirements presented in j), k), or l:
j) amino acid encoded by the above d), e) or f);
k) amino acids encoded by SEQ ID NO:4;
l) the above-mentioned amino acid sequence encoded by SEQ ID NO:4 can be deleted, replaced or inserted by one or more amino acids and the proteins encoded still possess the Fcf2 activity.

8. The method as defined in claim 2 wherein a new recombinant plasmid expressing reductase Fcf1 is constructed in Step (3) and cloned into pET28a (+) expression vector.

9. The method as defined in claim 2 wherein in Step (3) also directs that a recombinant strain is constructed to produce reductase Fcf1.

10. The method as defined in claim 2 wherein the plasmid pET28a (+) containing Fcf2 encoding sequence is constructed in Step (4).

11. The method as defined in claim 2 including constructing an expression strain containing Fcf2 to express target proteins in Step (4).

12. The use of dTDP-D-Fucofuranose of claim 1 wherein said dTDP-D-Fucofuranose acts as an anticancer drug.

13. The use of dTDP-D-Fucofuranose of claim 1 wherein said dTDP-D-Fucofuranose acts as goods or reaction substrates.

14. The method as defined in claim 4 wherein reductase Fcf1 have the same amino acids as the following g), h) or i):
g) amino acids encoded by a), b) or c);
h) amino acids encoded by SEQ ID NO:3;
i) the sequences derive from deletion, replacement or insertion of one or more amino acids of the amino acid sequences encoded by SEQ ID NO: 3 which can also produce active reductases.

15. The method as defined in claim 6 wherein the amino acid sequences of Fcf2 must meet the requirements presented in j), k), or l:
j) amino acid encoded by the above d), e) or f);
k) amino acids encoded by SEQ ID NO:4;
l) the above-mentioned amino acid sequence encoded by SEQ ID NO:4 can be deleted, replaced or inserted by one or more amino acids and the proteins encoded still possess the Fcf2 activity.


SEQUENCE LISTING (8 sequences)

SEQ ID NO: 1 - DNA, 951 bp, E. coli O52
atggatgctagaaaaaatggagtcctgattacaggcggtgctgggtttataggtaaagcattgatcaccgaaatggtcgaaaggcagatcccactagtgtcgttcgatatttcggataaaccagactcgttgcctgagctaagtgaatattttaactggtataaatttagctacctcgaatcttctcagcgtataaaagaactgcatgagatagttagccgccacaatattaagacagttattcatttggcaacaactatgtttccacatgaatcgaaaaaaaatatcgataaagattgtctggaaaatgtttatgcgaatgtctgttttttcaaaaacctatatgaaaatggttgtgagaaaattatatttgcatcatcgggtggcactgtttacggaaaatccgatacaccattttctgaagatgatgcacttttaccagagatcagttatggcttaagcaaagttatgactgagacatacttacgatttattgctaaagaactcaatggtaaatccatatcattgcggatctcaaatccttatggagaagggcagagaatagatggtaagcaaggtgtaataccaatatttttaaataaaatctcaaatgatattccaattgatattattggttcaattgagagtaaaagagattatatttatataagtgatcttgtacaagccttcatgtgttcattagaatatgaaggacatgaagatatttttaacataggttctggagaatctattacgttaaaaaaacttattgaaactatcgaatttaaattgaataaaaaggcagtaatcggttttcaagatcctatccataccaatgctaacggaattattttagatattaagcgagctatggctgagttaggatggcgtccgacggttgtactcgatgatggcattgataaattaataaaatcaattaggtgtaagtaa

SEQ ID NO: 2 - DNA, 1134 bp, E. coli O52
atgaataaggtccttatcattggtagtggatttagcggtgcaacaatagcaagactgttggctgaagagaatattaaagtaaagataatagacgacagaaagcatattggtggtaactgctatgatgagcgggatgagaaaacaggcattaatgtccacgtttatggaccgcatattttccatacggataatgaagatgtatggaattttgtgaataaatatgggacattccagccatatacaacgaggcttaaagcgaatgcaaaaggccagatttattcgttacctgtaaatcttcatacaattaatcaatactataaaacagcattgtctcctactgaagccaggaaactgattgcgagcaaaggtgatcagacaattaatgatcctcagtcttttgaagaacaagctttaaaatttgttggtgaagatttatacaaaacattcttttatggctatcctaaaaaacagtggggtatggagccaaaggaaataccagcatctgtattaaagcgtctcccagtacgttttaactatgatgataactattttttccataaattccagggcatacctcgtgatggctacacaccattatttcaaaatttactgaaccacccaaatatagagtttgaattaggaaagaaagtaaatagagcaactgttgaagaattaatcacctccgagcaatacggacatgtatttttctctggagcgattgatcatttctacgactatgaatttggcatgttgcaatatcgtacgctggattttgaaaagttttacagcgaagacgatgattatcagggttgtgttgtaatgtcttattgtgatgaagatgttccttatacccgcgtaacggaacataaatatttcacgccatgggaagagcataaaggaagcgtgttatacaaagagtttagtcgtagttgcgataaagaagatattccatattatccagttagacttgtctctggaaatagcatatggaacaaatatgaacaaaaggcaaaagaagagactaatataacattcatcgggcgtttggctacatatcgctatctcgatatggatgtctgtattaaagaagctattgaatgtgctcaattgtatataaagaataataaagaatga

SEQ ID NO: 3 - protein, 316 aa, E. coli O52
MDARKNGVLITGGAGFIGKALITEMVERQIPLVSFDISDKPDSLPELSEYFNWYKFSYLESSQRIKELHEIVSRHNIKTVIHLATTMFPHESKKNIDKDCLENVYANVCFFKNLYENGCEKIIFASSGGTVYGKSDTPFSEDDALLPEISYGLSKVMTETYLRFIAKELNGKSISLRISNPYGEGQRIDGKQGVIPIFLNKISNDIPIDIIGSIESKRDYIYISDLVQAFMCSLEYEGHEDIFNIGSGESITLKKLIETIEFKLNKKAVIGFQDPIHTNANGIILDIKRAMAELGWRPTVVLDDGIDKLIKSIRCK

SEQ ID NO: 4 - protein, 377 aa, E. coli O52
MNKVLIIGSGFSGATIARLLAEENIKVKIIDDRKHIGGNCYDERDEKTGINVHVYGPHIFHTDNEDVWNFVNKYGTFQPYTTRLKANAKGQIYSLPVNLHTINQYYKTALSPTEARKLIASKGDQTINDPQSFEEQALKFVGEDLYKTFFYGYPKKQWGMEPKEIPASVLKRLPVRFNYDDNYFFHKFQGIPRDGYTPLFQNLLNHPNIEFELGKKVNRATVEELITSEQYGHVFFSGAIDHFYDYEFGMLQYRTLDFEKFYSEDDDYQGCVVMSYCDEDVPYTRVTEHKYFTPWEEHKGSVLYKEFSRSCDKEDIPYYPVRLVSGNSIWNKYEQKAKEETNITFIGRLATYRYLDMDVCIKEAIECAQLYIKNNKE

SEQ ID NO: 5 - DNA, 32 bp, Artificial sequence
  synthesized
cggaattcatggatgctagaaaaaatggagtc

SEQ ID NO: 6 - DNA, 33 bp, Artificial sequence
  synthesized
ccgctcgagttacttacacctaattgattttat

SEQ ID NO: 7 - DNA, 33 bp, Artificial sequence
  synthesized
cggaattcatgaataaggtccttatcattggta

SEQ ID NO: 8 - DNA, 33 bp, Artificial sequence
  synthesized
ccgctcgagtcattctttattattctttatata